(19) United States
(12) Patent Application Publication
(10) Pub. No.: US 2017/0311928 A1
(43) Pub. Date: Nov. 2, 2017

(54) ULTRASONIC IMAGING APPARATUS AND METHOD OF CONTROLLING THE SAME

(71) Applicant: SAMSUNG ELECTRONICS CO., LTD., Suwon-si (KR)

(72) Inventors: Tae Ho JEON, Seoul (KR); Jong Keun SONG, Yongin-si (KR)

(73) Assignee: SAMSUNG ELECTRONICS CO., LTD., Suwon-si (KR)

(21) Appl. No.: 15/525,221

(22) PCT Filed: Nov. 7, 2014
(86) PCT No.: PCT/KR2014/010689

§ 371 Date: May 8, 2017

(51) Int. Cl. A61B 8/00; A61B 8/08; A61B 8/14

(52) U.S. Cl. CPC A61B 8/4494; A61B 8/54; A61B 8/5269; A61B 8/5207; A61B 8/14; A61B 8/461; A61B 8/4405; A61B 8/467

(57) ABSTRACT

A display apparatus and driving method are discussed where the ultrasonic imaging apparatus includes an ultrasonic transducer configured to sequentially output a first ultrasonic signal, a second ultrasonic signal, and a third ultrasonic signal; a beamforming unit configured to generate a first synthesized signal by delaying the first ultrasonic signal and synthesizing the delayed first ultrasonic signal with the second ultrasonic signal, and to generate a second synthesized signal by delaying the first synthesized signal and synthesizing the delayed first synthesized signal with the third ultrasonic signal.

### TECHNICAL FIELD

The present disclosure relates to an ultrasonic imaging apparatus of generating ultrasound images, and a control method thereof.

### BACKGROUND ART

An ultrasonic imaging apparatus is medical equipment of acquiring slice images (for example, slice images of soft tissues or images of blood flow) of an object, for example, various tissues or structures in a human body using ultrasonic waves. The ultrasonic imaging apparatus has advantages that it is a compact, low-priced apparatus compared to other medical imaging apparatuses, and it can display images in real time. Also, the ultrasonic imaging apparatus has high safety since there is no risk for patients to be exposed to X-rays. For the advantages, the ultrasonic imaging apparatus is widely used to diagnose the heart, breasts, abdomen, urinary organs, uterus, etc.

The ultrasonic imaging apparatus irradiates ultrasonic waves toward a target region inside an object through a plurality of channels, collects echo ultrasonic waves reflected from the target region through the plurality of channels, and then generates an ultrasound image based on information of the collected ultrasonic waves. For this, the ultrasonic imaging apparatus performs beamforming to estimate the magnitudes of reflected waves in specific space from signals output through the plurality of channels, and creates an ultrasound image based on the beamformed signals. The beamforming corrects the time differences of signals output through the plurality of channels, and applies weights to the signals with the corrected time differences, thereby focusing ultrasonic signals.

### DISCLOSURE

### Technical Problem

An object of the present invention is to provide an ultrasonic imaging apparatus of performing beamforming sequentially, and a control method thereof.

### Technical Solution

In accordance with one aspect of the present disclosure, an Ultrasonic imaging apparatus comprising: an ultrasonic transducer configured to sequentially output a first ultrasonic signal, a second ultrasonic signal, and a third ultrasonic signal; a beamforming unit configured to generate a first synthesized signal by delaying the first ultrasonic signal and synthesizing the delayed first ultrasonic signal with the second ultrasonic signal, and to generate a second synthesized signal by delaying the first synthesized signal and synthesizing the delayed first synthesized signal with the third ultrasonic signal.

The first ultrasonic signal may be delayed until the second ultrasonic signal is output, and the first synthesized signal is delayed until the third ultrasonic signal is output.

The beamforming unit may amplify at least one ultrasonic signal of the first ultrasonic signal, the second ultrasonic signal, and the third ultrasonic signal.

The beamforming unit may control the output of the delayed first synthesized signal so that the delayed first synthesized signal is synthesized with the third ultrasonic signal.

The beamforming unit may comprise: a first synthesizer configured to generate the first synthesized signal by synthesizing the delayed first ultrasonic signal with the second ultrasonic signal; a first delay configured to delay and output the first synthesized signal; and a second synthesizer configured to generate the second synthesized signal by synthesizing the delayed first synthesized signal with the third ultrasonic signal.

The ultrasonic imaging apparatus may further comprising a switch configured to output the delayed first synthesized signal to the second synthesizer.

The first synthesizer and the second synthesizer may generate the first synthesized signal and the second synthesized signal, respectively, by applying a weight value.

An Ultrasonic imaging apparatus comprising: an ultrasonic transducer configured to output a first ultrasonic signal and a second ultrasonic signal; and a beamforming unit configured to delay and output a first synthesized signal generated using the first ultrasonic signal, and to generate a second synthesized signal by synthesizing the delayed first synthesized signal with the second ultrasonic signal.

The first ultrasonic signal may be at least one ultrasonic signal output earlier than the second ultrasonic signal.

The first synthesized signal may be generated by synthesizing a plurality of first ultrasonic signals output earlier than the second ultrasonic signal

The beamforming unit may comprise: a delay unit configured to delay the first ultrasonic signal or the first synthesized signal, and to output the delayed signal; and

a synthesizer configured to synthesize the delayed signal with the second ultrasonic signal to generate the second synthesized signal.

The synthesizer may synthesize the delayed signal with the second ultrasonic signal by applying a weight.

The delay may delay the first ultrasonic signal or the second ultrasonic signal until the second ultrasonic signal is output

The beamforming unit may further comprise a switch configured to decide a synthesizer to which the delay signal is input.

The beamforming unit may further comprise an amplifier to amplify the second ultrasonic signal.

The beamforming unit may further comprise a filter configured to remove noise from the second ultrasonic signal.

A method of controlling an ultrasonic imaging apparatus, the method comprising: delay operation of synthesizing a delayed first ultrasonic signal with a second ultrasonic signal to generate a first synthesized signal, and delaying the first synthesized signal; and synthesis operation of synthesizing the delayed first synthesized signal with a third ultrasonic signal to generate a second synthesized signal.

The delay operation may comprise delaying the first synthesized signal until the third ultrasonic signal is output.

The synthesis operation may comprises applying a weight to the first synthesized signal and the third ultrasonic signal.

The synthesis operation may further comprise deciding the weight that is to be applied to the first synthesized signal and the third ultrasonic signal.

The method may further comprising controlling the output of the first synthesized signal so that the delayed first synthesized signal is synthesized with the third ultrasonic signal

### Advantageous Effects

It is possible to miniaturize circuit for beam forming by providing an ultrasonic imaging apparatus of performing beamforming sequentially, and a control method thereof.

### DESCRIPTION OF DRAWINGS

FIG. 1 is a perspective view of an ultrasonic imaging apparatus according to an embodiment of the present invention.

FIG. 2 is a control block diagram of an ultrasonic imaging apparatus according to an embodiment.

FIG. 3 is a plan view for explaining an embodiment of the ultrasound probe.

FIG. 4 is a diagram for explaining a delay of an ultrasonic signal.

FIG. 5 is a control block diagram for explaining a beamforming unit according to an embodiment.

FIG. 6 is a control block diagram for explaining a signal processing unit according to an embodiment.

FIG. 7 is a circuit diagram for explaining the delay unit in detail.

FIG. 8 is a control block for explaining an example of the ultrasonic signal processing of the beamforming unit.

FIG. 9 is a flowchart illustrating a method of controlling an ultrasonic imaging apparatus according to an embodiment of the present invention.

### MODE FOR INVENTION

Hereinafter, embodiments of the present disclosure will be described in detail with reference to the appended drawings such that one of ordinary skill in the art can easily understand and embody the present disclosure.

In the following description, a detailed description of known functions and configurations incorporated herein will be omitted when it may make the subject matter of the present invention rather unclear. Further, terms used in the embodiments as described below are defined in consideration of functions in the embodiments, and the meanings of the terms may vary depending on a user's or operator's intention or practice. Therefore, the terms used in the embodiments should be interpreted based on the definition in the specification, and unless specifically defined, the terms are interpreted as common meanings of the terminologies that one of ordinary skill in the art to which the present disclosure pertains understands.

Also, in the following description, aspects described optionally or configurations of embodiments described optionally must be construed as being able to be freely combined with each other, if not specified, although they are shown as a single integrated configuration in the drawings, unless the combination is clearly technical contradiction as determined by one of ordinary skill in the art.

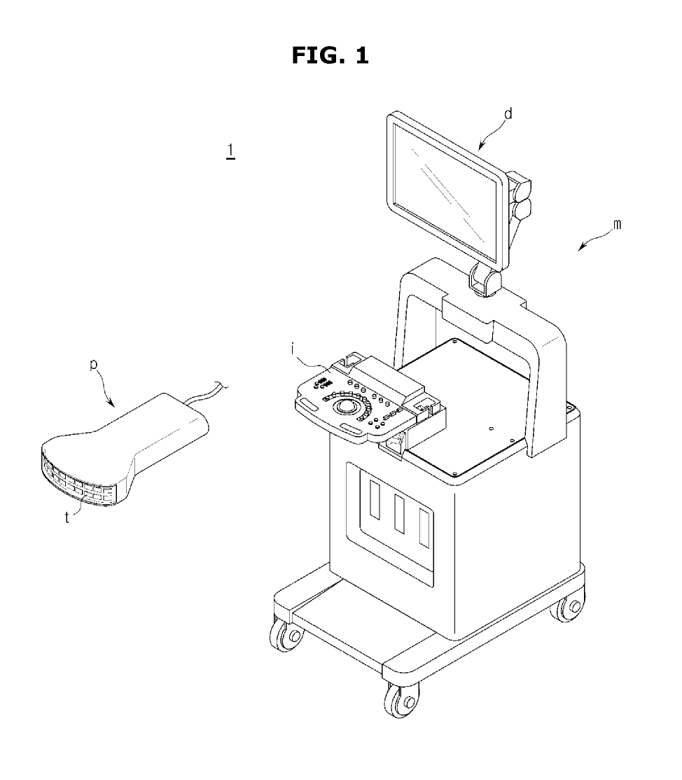

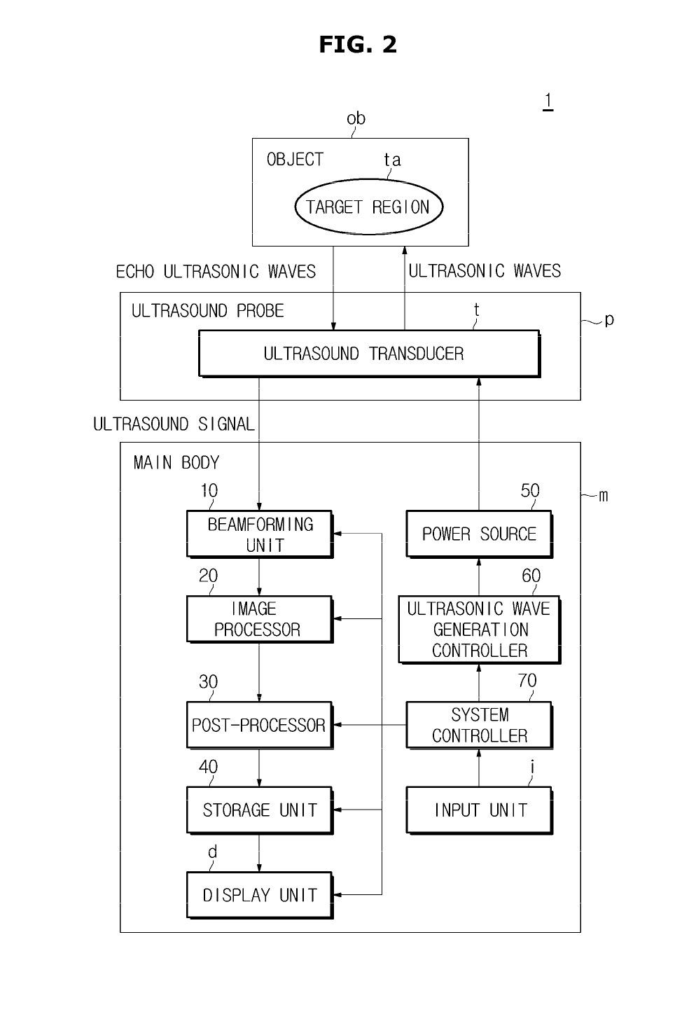

FIG. 1 is a perspective view of an ultrasonic imaging apparatus according to an embodiment of the present disclosure. FIG. 2 is a control block diagram of an ultrasonic imaging apparatus according to an embodiment of the present disclosure.

As illustrated in FIG. 1, an ultrasonic imaging apparatus 1 may include an ultrasound probe p configured to emit ultrasonic waves onto an object, to receive echo ultrasonic waves from the object, and to output ultrasonic signals corresponding to the received echo ultrasonic waves, and a main body m configured to generate an ultrasound image based on the ultrasonic signals received from the ultrasound probe p.

For convenience of description, it is assumed that the ultrasound probe p outputs only ultrasonic signals, and the main body m performs beamforming to create an ultrasound image, although the present disclosure is not limited to this. According to other examples, the ultrasound probe p may perform beamforming and transfer the results of the beamforming to the main body m, or the ultrasound probe p may perform beamforming to create an ultrasound image based on the result of the beamforming.

As shown in FIG. 1, the main body m may be a workstation connected to the ultrasound probe p and having an input unit i and a display unit d, although not limited to this. In the following description, for convenience of description, it is assumed that the main body m is provided with the input unit i and the display unit d.

The input unit i may receive a predetermined instruction or command from a user in order to control the ultrasonic imaging apparatus 1. For example, the input unit i may receive various commands for creating ultrasound images, and commands for post-processing of the created ultrasound images.

The input unit i may include a user interface, such as, for example, a keyboard, a mouse, a trackball, a touch screen, or a paddle, although not limited to this.

The display unit i may display ultrasound images on the screen. For example, the display unit i may display two-dimensional or three-dimensional ultrasound images. Also, the display unit i may display, as well as ultrasound images, various information processed by the ultrasonic imaging apparatus 1 on the screen through a Graphic User Interface (GUI). According to another embodiment, the ultrasonic imaging apparatus 1 may include two or more display units.

The display unit i may be a Cathode Ray Tube (CRT) display, a Liquid Crystal Display (LCD), an Organic Light Emitting Diode (OLED) display, or the like, although not limited to these. Hereinafter, the individual components of the ultrasonic imaging apparatus 1 will be described in detail.

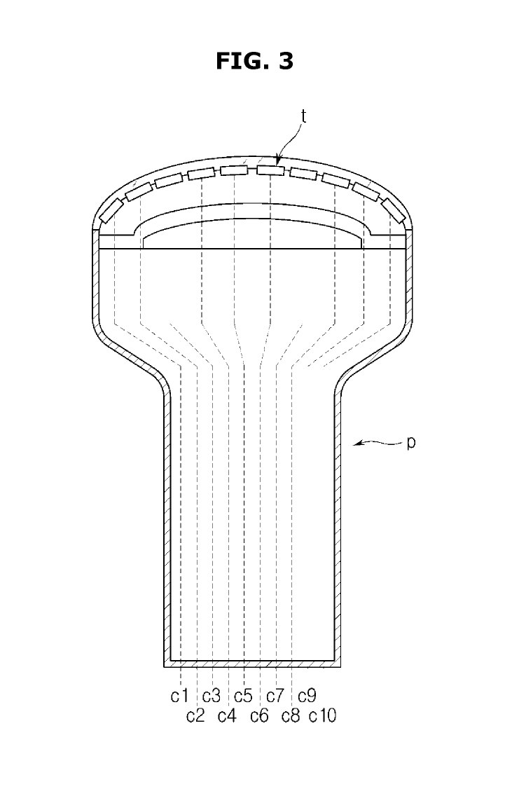

FIG. 3 is a plan view for describing an ultrasound probe according to an embodiment of the present disclosure.

As shown in FIG. 1, the ultrasound probe p may include a plurality of transducers, wherein the plurality of transducers may be arranged two-dimensionally. The ultrasonic imaging apparatus 1 may create three-dimensional ultrasound images based on ultrasonic signals output from the transducers arranged two-dimensionally.

Referring to FIG. 3, the ultrasound probe p may be provided with a plurality of ultrasonic transducers t at one end. The ultrasonic transducers t may generate ultrasonic waves according to an applied signal or voltage, irradiate the ultrasonic waves to an object, receive echo ultrasonic waves reflected from the object, and thus generate and output ultrasonic signals.

More specifically, the ultrasonic transducers t may receive a voltage from an external power source 50 or an internal condenser such as a battery, and vibrate piezoelectric vibrators or thin films according to the received voltage to generate ultrasonic waves. Also, the ultrasonic transducers t may vibrate the piezoelectric material or the thin films when receiving echo ultrasonic waves to generate alternating current of a frequency corresponding to the vibration frequency, thereby converting the received echo ultrasonic waves into ultrasonic signals. The ultrasonic signals may be transferred to a beamforming unit 10. Each ultrasonic transducer t may be a Magnetostrictive Ultrasonic Transducer (MUT) using a magnetostrictive effect of magnetic materials, a Piezoelectric Ultrasonic Transducer (PUT) using a piezoelectric effect of piezoelectric materials, or a Capacitive Micromachined Ultrasonic Transducer (CMUT) transmitting/receiving ultrasonic waves using vibrations of several hundreds or thousands of micromachined thin films. However, the ultrasonic transducer t may be any type of transducer capable of generating ultrasonic waves according to an electrical signal or generating an electrical signal according to ultrasonic waves.

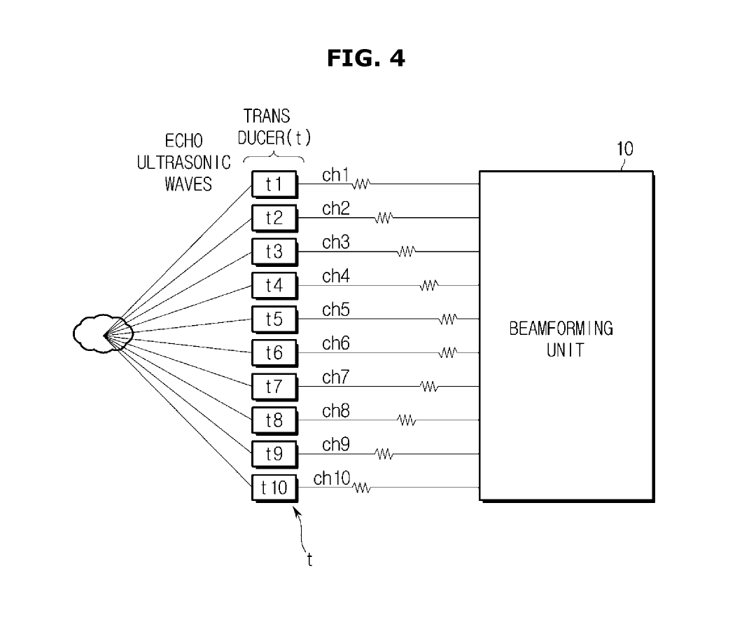

Hereinafter, the beamforming unit 10 will be described in detail with reference to FIGS. 4 to 7

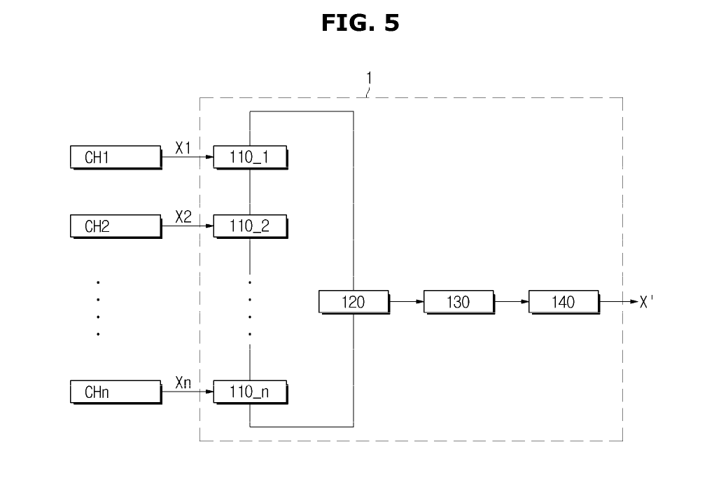

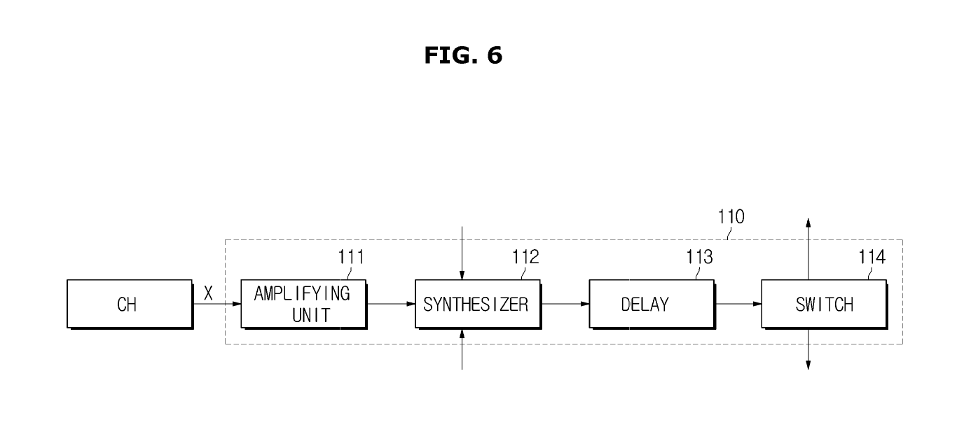

FIG. 4 is a view for describing delays of ultrasonic signals. FIG. 5 is a control block diagram for describing a beamforming unit according to an embodiment of the present disclosure. FIG. 6 is a control block diagram for describing a signal processor according to an embodiment of the present disclosure.

As shown in FIG. 4, the ultrasound probe (p of FIG. 3) may be connected to the beamforming unit 10 through a plurality of channels. Herein, each channel means a channel for transmitting an ultrasonic signal output from an ultrasonic transducer to the beamforming unit 10, and the plurality of transducers t may transmit ultrasonic signals X1 to X10 to the beamforming unit 10 through the respective channels.

At this time, the ultrasonic signals X1 to X10 output from the respective channels may be sequentially input to the beamforming unit 10 with predetermined time differences. More specifically, the time differences may be caused due to differences in distance between the transducers t and a target region of an object. The transducers close to the target region may receive echo ultrasonic waves early, and the transducers located distant from the target region may receive the echo ultrasonic waves later. As such, since the transducers t receive echo ultrasonic waves with time differences, ultrasonic signals output from the channels may also have predetermined time differences.

The beamforming unit 10 may perform beamforming on a plurality of ultrasonic signals input with the time differences. For this, if ultrasonic signals are sequentially output from the plurality of channels, the beamforming unit 10 may synthesize and delay the sequentially outputted ultrasonic signals sequentially, thereby performing beamforming. More specifically, the beamforming unit 10 may perform beamforming by repeatedly performing signal processing of delaying a first ultrasonic signal until a second ultrasonic signal is output, and synthesizing the delayed first ultrasonic signal with the second ultrasonic signal when the second ultrasonic signal is output to generate a synthesized signal.

The first ultrasonic signal represents an ultrasonic signal input to the beamforming unit 10 before the second ultrasonic signal is input. The first ultrasonic signal may be an ultrasonic signal output from one channel, or a synthesized ultrasonic signal of a plurality of ultrasonic signals output from the plurality of channels.

As shown FIG. 5, the beamforming unit 10 includes a plurality of signal processors 110_1 to 110_n configured to synthesize and delay ultrasonic signals, a filter 130 configured to remove noise from signals output from the signal processors 110_1 and 110_n, a signal storage unit 120 configured to store the signals from which noise has been removed, and a stored signal synthesizer 140 configured to synthesize the signals stored in the signal storage unit 120.

The signal processors 110_1 to 110_n may delay ultrasonic signals. In addition, the signal processors 110_1 to 110_n may synthesize ultrasonic signals. More specifically, the signal processors 110_1 to 110_n may be provided to correspond to the respective channels C1 to Cn to delay and output an ultrasonic signal output from each of the channels C1 to Cn, or to synthesize ultrasonic signals output from the different channels C1 to Cn.

The plurality of signal processors 110_1 to 110_n may be connected to each other. For example, neighboring ones of the signal processors 110_1 to 110_n may be connected to each other to transmit/receive ultrasonic signals.

The signal storage unit 120 may store ultrasonic signals output from a part of the signal processors 110_1 and 110_n. For this purpose, the signal storage unit 120 may be connected to some signal processors 110_1 to 110_n among the plurality of signal processors 110_1 to 110_n. For example, the signal storage unit 120 may be embodied as a device such as a buffer capable of temporarily storing ultrasonic signals, to store ultrasonic signals output from the signal processors 110_1 and 110_n. In FIG. 5, the beamforming unit 10 includes a single signal storage unit 120, however, the beamforming unit 10 may include a plurality of signal storage units 120.

The filter 130 may remove noise from ultrasonic signals to obtain a clearer ultrasound image. At this time, the filter 130 may be disposed between the signal processors 110_1 and 110_n and the signal storage unit 120, although not limited to this. For example, the filter 130 may be disposed between the signal storage unit 120 and a focusing device. Also, the filter 130 may be included in each of the signal processors 110_1 to 110_n.

The stored signal synthesizer 140 may synthesize the ultrasonic signals stored in the signal storage unit 120. At this time, the stored signal synthesizer 140 may synthesize ultrasonic signals output from a part of the signal processors 110_1 to 110_n and stored in the signal storage unit 120.

Hereinafter, the configuration of each signal processor 110 will be described in detail with reference to FIG. 6. As shown in FIG. 6, the signal processor 110 may include an amplifying unit 111 configured to amplify ultrasonic signals, a synthesizer 112 configured to synthesize ultrasonic signals, a delay unit 113 configured to delay ultrasonic signals, and a switch 114 configured to adjust the outputs of ultrasonic signals.

The amplifying unit 111 can amplify and output a signal received from each channel. For this, the amplifying unit 111 may include an amplifier. For example, the amplifying unit 111 may include a low noise amplifier (LNA) for reducing noise of a signal received from each channel, a variable gain amplifier (VGA) for adjusting gain according to a received signal, or a pre-amplifier. The VGA may be Time Gain Compensation (TGC) to compensate gain according to a distance between a target region and the transducer, although not limited to this.

The synthesizer 112 may receive one or more ultrasonic signals, synthesize the received ultrasonic signals, and then output a synthesized signal. At this time, the synthesizer 112 may apply a predetermined weight to the received signals according to a control signal, and then synthesize the signals. By applying a predetermined weight to the received signals, the ultrasonic signals received from the respective channels can be relatively emphasized or relatively attenuated

In this case, the predetermined weight may be decided by data-independent beamforming (or fixed beamforming), or data-dependant beamforming (or adaptive beamforming).

The data-independent beamforming, which is also called fixed beamforming, is to perform beamforming by applying the same predetermined weight to each input data regardless of the kind of the data.

The adaptive beamforming is to perform beamforming by applying different weights to input data. In the adaptive beamforming, beamforming is performed by deciding an optimal weight corresponding to input data according to the input data, and then applying the decided weight to the input data.

Meanwhile, the predetermined weight may have been stored in advance in the storage unit 40, or may be calculated by a controller.

The delay unit 113 may delay an input signal by a predetermined time period according to a control signal. Accordingly, the delay unit 113 may include a delay to delay ultrasonic signals. A delay time period by which a signal input to the delay unit 113 is delayed may be fixed, but the delay time period may be adjusted according to a control signal.

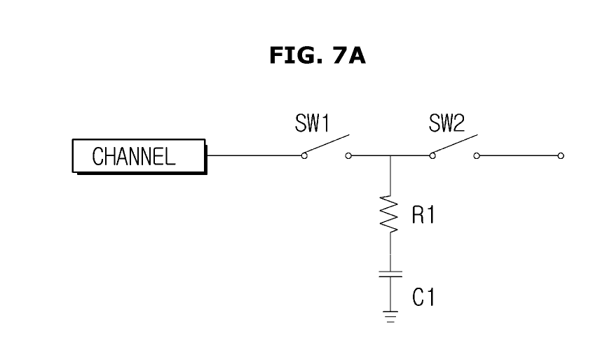

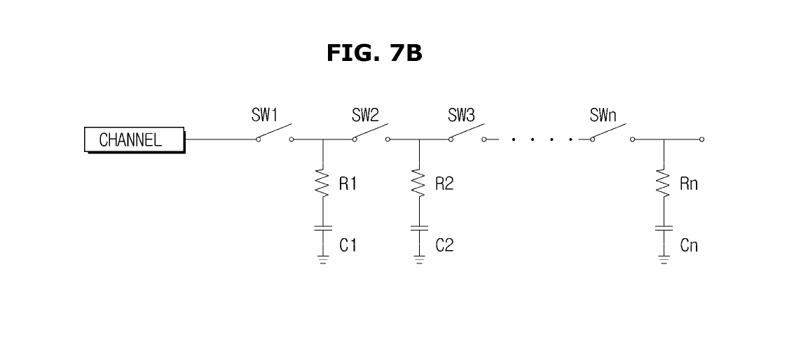

Hereinafter, an example of the delay unit 113 will be described in detail with reference to FIG. 7

FIG. 7 is a circuit diagram for describing a delay unit in detail. The delay unit 113 may include a delay. The circuit of FIG. 7 is an example of a delay circuit to delay an input signal by a predetermined time delay.

As shown in FIG. 7A, the delay circuit may be configured with first and second switches SW1 and SW2, a resistor R1, and a capacitor C1.

As shown in FIG. 7A, if the first switch SW1 is turned on according to a control signal, an ultrasonic signal input through a channel may be stored in the capacitor C. If the first switch SW1 is turned off, and the second switch SW2 is turned on, the ultrasonic signal stored in the capacitor C1 may be output.

More specifically, an ultrasonic signal output from each channel may be an electrical signal corresponding to ultrasonic waves received by each transducer. Therefore, if the first switch SW1 is turned on, a predetermined voltage may be applied to the resistor R1, and charges may be charged in the capacitor C by the applied voltage. Also, if the first switch SW1 is turned off, and the second switch SW2 is turned on, the charges charged in the capacitor (C) may be output toward the second switch SW2. If operation of charging and discharging the capacitor C in this way is repeatedly performed at short time intervals, an ultrasonic signal output from each channel may become equal to an electric signal output by the charged charges of the capacitor C.

In order to increase a delay time period of an ultrasonic signal, there is a method of increasing a charging and discharging period of the capacitor C. However, as the charging and discharging period becomes longer, a degree of similarity between an ultrasonic signal and an electrical signal output by charges charged in the capacitor C may be reduced.

Therefore, in order to increase a delay time period, as shown in FIG. 7B, the delay may include a plurality of switches SW1 to SWn, a plurality of resistors R1 to Rn, and a plurality of capacitors C1 to Cn. More specifically, the plurality of switches SW1 to SWn may be controlled so that ultrasonic signals output from the channels are sequentially delayed by the first to n-th capacitors C1 to Cn. FIG. 7 illustrates the delay unit 113 in detail. However, the delay unit 113 is not limited to the example of FIG. 7. That is, the delay unit 113 may be any circuit that can delay an input signal by a predetermined time period.

The switch 114 may adjust the output of an ultrasonic signal delayed by the delay unit 113 according to a control signal. As described above, the plurality of signal processors may be connected to each other. A signal processor to which an ultrasonic signal delayed by the delay unit 113 is to be input may be decided by the switch 114. At this time, the switch 114 may cause a delayed ultrasonic signal to be input to a single signal processor or to the plurality of signal processors.

Referring again to FIG. 2, the image processor 20 may image the inside of an object (for example, a human body) based on an ultrasonic signal beamformed by the beamforming unit 10 so that a user (for example, a doctor or a patient) can visually examine the inside of the object. That is, the image processor 20 may create an ultrasound image using an ultrasonic signal beamformed by the beamforming unit 10, and transfer the ultrasound image to the storage unit 40 or the display unit d.

The post-processor 30 may perform additional processing on the image created by the image processing unit 20. For example, the post-processor 30 may perform image post-processing such as correcting or re-adjusting the contrast, brightness or sharpness of the ultrasound image. Also, the post-processor 30 may emphasize a specific region of the ultrasound image as necessary. Also, the post-processor 30 may create a plurality of ultrasound images, and then create a stereoscopic image using the plurality of ultrasound images. The additional image processing of the post-processor 30 may be performed according to predetermined settings or according to a user's instruction or command input through the input unit i.

The storage unit 40 may store various information required for creating an ultrasound image. Also, the storage unit 40 may store the ultrasound image created by the image processor 20 or the ultrasound image post-processed by the post-processor 30, and display the ultrasound image on the display unit d according to a request from a user or the like. At this time, the storage unit 40 may be implemented as various kinds of storage medium, such as a flash memory, a hard disk, and Electrically Erasable and Programmable Read Only Memory (EEPROM).

An ultrasonic wave generation controller 60 may generate a pulse signal according to a command from the system controller 70, and transfer the pulse signal to the ultrasound probe p so that the ultrasonic transducers t generate ultrasonic waves in accordance with the pulse signal, and irradiate the ultrasonic waves to an object. The power source 50 may apply predetermined alternating current to the ultrasound probe p according to the control of the ultrasonic wave generation controller 60.

The system controller 70 may control overall operations of the ultrasonic imaging apparatus 1 including the beamforming unit 10, the image processor 20, the post-processor 30, the storage unit 40, the ultrasonic wave generation controller 60, and the display unit d. According to another embodiment, the system controller 70 may control the operations of the ultrasonic imaging apparatus 1 according to predetermined settings, or may generate a predetermined control command in accordance with a user's instruction or command input through the input unit i and then control the operations of the ultrasonic imaging apparatus 1.

In addition, the system controller 70 may generate a control signal, and control the beamforming unit 10 to beamform ultrasonic signals sequentially input. Hereinafter, an example of ultrasonic signal processing of the system controller 70 will be described in detail with reference to FIG. 7

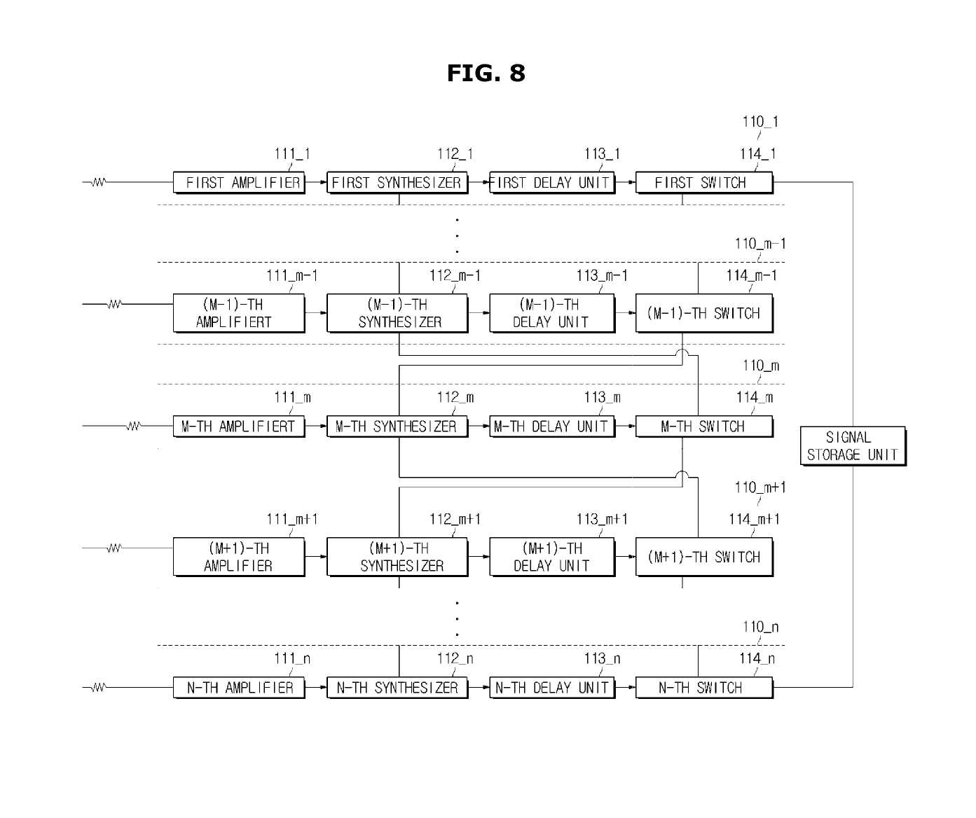

FIG. 8 is a control block for describing an example of ultrasonic signal processing of the beamforming unit. As shown in FIG. 8, ultrasonic signals may be sequentially output through n channels from a m-th channel. At this time, the m-th channel from which an ultrasonic signal is output first may be the channel of a transducer located closest to a target region.

The controller may repeatedly perform operation of delaying and synthesizing a plurality of ultrasonic signals output sequentially. More specifically, the controller may control the beamforming unit 10 to repeatedly perform signal processing of delaying a first ultrasonic signal until a second ultrasonic signal is received, and then synthesizing the first ultrasonic signal with the second ultrasonic signal when the second ultrasonic signal is received to generate a synthesized signal. Herein, the first ultrasonic signal represents an ultrasonic signal output earlier than the second ultrasonic signal, and the first ultrasonic signal may be a synthesized signal generated by synthesizing a delayed ultrasonic signal with an output ultrasonic signal. That is, the first ultrasonic signal and the second ultrasonic signal are relative concepts. When another ultrasonic signal is input later than the second ultrasonic signal, and the delayed first ultrasonic signal and the second ultrasonic signal are synthesized to generate a synthesized signal, the synthesized signal becomes the first ultrasonic signal, and the ultrasonic signal input later than the second ultrasonic signal becomes the second ultrasonic signal.

Referring again to FIG. 7, when a predetermined time elapses after an ultrasonic signal is output from the m-th channel, ultrasonic signals Xm may be output from (m+1)-th and (m−1)-th channels adjacent to the m-th channel. When a predetermined time elapses after the ultrasonic signals Xm are output from the (m+1)-th and (m−1)-th channels, ultrasonic signals may be output from (m+2)-th and (m−2)-th channels. In this way, ultrasonic signals may be sequentially output from the n channels.

Accordingly, the controller can control the beamforming unit 10 by generating control signals such that the ultrasonic signals sequentially output are sequentially delayed and synthesized.

More specifically, the ultrasonic signal output from the m-th channel may be amplified by a m-th amplifier 111_m, and input to a m-th synthesizer 112_m. At this time, the m-th synthesizer 112_m may output the ultrasonic signal as it is according to a control signal. Thus, the ultrasonic signal output from the m-th synthesizer 112_m may be delayed by the m-th delay 113_m by a predetermined time period, and then output. At this time, the delay time period may vary according to a control signal. For example, the m-th delay 113_m may delay and output the ultrasonic signal until ultrasonic signals are output from the (m+1)-th and (m−1)-th channels.

When ultrasonic signals are output from the (m+1)-th and (m−1)-th channels, the respective ultrasonic signals may be amplified by the respective amplifiers 111_m+1 and 111_m−1 and input to the respective synthesizers 112_m+1 and 112_m−1. At this time, as described above, the ultrasonic signal delayed by the m-th delay 113_m may be input to the respective synthesizers 111_m+1 and 111_m−1 together with the ultrasonic signals amplified by the respective amplifiers 111_m+1, 111_m−1, synthesized by the respective synthesizers 111_m+1 and 111_m−1, and then output. At this time, the synthesizers 112_m+1 and 112_m−1 may apply a predetermined weight according to a control signal.

As such, the ultrasonic signals output by the respective synthesizers 112_m+1 and 112_m−1 may be delayed by the delays 113_m+1 and 113_m−1 by a predetermined time period, and then output. At this time, the switches 114_m+1 and 114_m−1 can output the ultrasonic signals output from the delays 113_m+1 and 113_m−1 only in the direction in which the ultrasonic signals are input, according to a control signal.

The controller may control the beamforming unit 10 to repeat the signal processing as described above. Accordingly, the controller may synthesize a synthesized signal of ultrasonic signals output before an ultrasonic signal is output from the first channel with the ultrasonic signal output from the first channel, and store the synthesized signal in the signal storage unit 120. Also, the controller may synthesize a synthesized signal of ultrasonic signals output before an ultrasonic signal is output from the n-th channel with the ultrasonic signal output from the n-th channel, and store the synthesized signal in the signal storage unit 120.

If the two ultrasonic signals are stored in the signal storage unit 120, the stored ultrasonic signals may be filtered, and the filtered ultrasonic signals may be focused to generate a beamforming signal.

The area of the beamforming unit 10 can be reduced by performing beamforming in such a way to delay and synthesize ultrasonic signals. More specifically, the time difference between ultrasonic signals output from the individual channels may increase in proportion to the number n of the channels, and the size of the delay unit 113 may also increase in proportional to the time difference. Therefore, if an ultrasonic signal is corrected after correcting the time difference input from each channel, the size of each delay unit 113 may increase in proportion to n. However, when delay and synthesis are repeated, each delay unit 113 may need to correct only the time difference between the adjacent channels. Therefore, the delay unit 113 may have the same size.

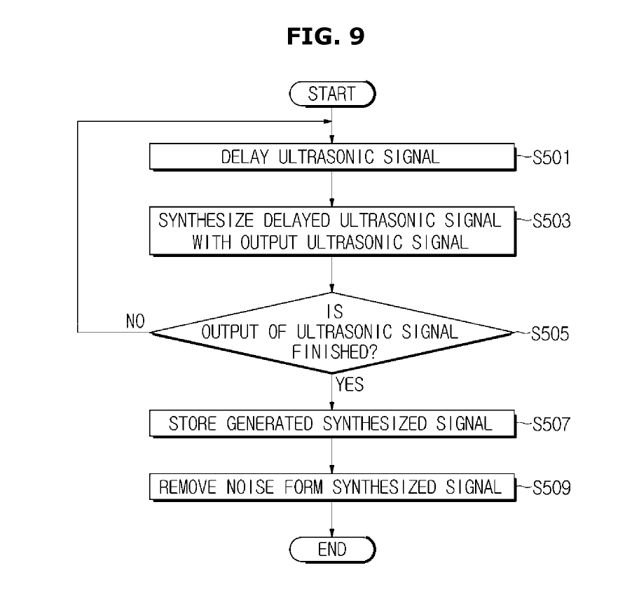

FIG. 9 is a flowchart illustrating a method of controlling an ultrasonic imaging apparatus according to an embodiment of the present invention.

Referring to FIG. 9, an ultrasonic signal is delayed, in operation S501. The delayed ultrasonic signal may be an ultrasonic signal output from any one of the plurality of channels, or a synthesized signal generated by synthesizing ultrasonic signals output from the plurality of channels. More specifically, the ultrasonic signal may be delayed until the next ultrasonic signal is input. The delayed ultrasonic signal may be output to the synthesizer 112 to which the next ultrasonic signal is input, among the plurality of synthesizers 112.

Then, the delayed ultrasonic signal and an output ultrasonic signal may be synthesized, in operation S503. At this time, the output ultrasonic signal may be amplified before it is synthesized. More specifically, when an ultrasonic signal is output from another one of the plurality of channels, the delayed ultrasonic signal and the output ultrasonic signal may be synthesized to generate a synthesized signal. At this time, the synthesized signal may be relatively emphasized or relatively attenuated by applying a predetermined weight to the delayed ultrasonic signal and the output ultrasonic signal. For the operation, operation of applying a weight may be further performed.

Thereafter, it is determined whether the output of ultrasonic signals is finished, in operation S505. More specifically, if the output of ultrasonic signals from the channels is not yet finished (NO in operation S505), the synthesized signal may be delayed until the next ultrasonic signal is output, in operation S501.

On the other hand, if the output of ultrasonic signal from the channels is finished (YES in operation S501), the synthesized signal may be stored, in operation S505, and noise may be removed from the stored synthesized signal, in operation S507. More specifically, the synthesized signal may pass through a predetermined filter to remove a signal of a specific band. On the other hand, noise may be first removed from the synthesized signal, and then the synthesized signal from which noise has been removed may be stored.

According to the ultrasonic imaging apparatus and the control method thereof as described above, a circuit for beamforming can be miniaturized.

It will be apparent to those skilled in the art that various modifications, substitutions, and alterations can be made hereto without departing from the spirit and scope of the invention as defined by the appended claims. Therefore, the embodiments and the accompanying drawings described above are intended to illustrate and not limit the technical idea, and the scope of technical thought is not limited by these embodiments and the accompanying drawings. The scope of which is to be interpreted by the following claims, and all technical ideas which are within the scope of the same shall be construed as being included in the scope of the right.

1. An Ultrasonic imaging apparatus comprising:
an ultrasonic transducer configured to sequentially output a first ultrasonic signal, a second ultrasonic signal, and a third ultrasonic signal;
a beamforming unit configured to generate a first synthesized signal by delaying the first ultrasonic signal and synthesizing the delayed first ultrasonic signal with the second ultrasonic signal, and to generate a second synthesized signal by delaying the first synthesized signal and synthesizing the delayed first synthesized signal with the third ultrasonic signal.

2. The ultrasonic imaging apparatus according to claim 1, wherein the first ultrasonic signal is delayed until the second ultrasonic signal is output, and the first synthesized signal is delayed until the third ultrasonic signal is output.

3. The ultrasonic imaging apparatus according to claim 1, wherein the beamforming unit amplifies at least one ultrasonic signal of the first ultrasonic signal, the second ultrasonic signal, and the third ultrasonic signal.

4. The ultrasonic imaging apparatus according to claim 1, wherein the beamforming unit controls the output of the delayed first synthesized signal so that the delayed first synthesized signal is synthesized with the third ultrasonic signal

5. The ultrasonic imaging apparatus according to claim 1, wherein the beamforming unit comprises:
a first synthesizer configured to generate the first synthesized signal by synthesizing the delayed first ultrasonic signal with the second ultrasonic signal;
a first delay configured to delay and output the first synthesized signal; and
a second synthesizer configured to generate the second synthesized signal by synthesizing the delayed first synthesized signal with the third ultrasonic signal.

6. The ultrasonic imaging apparatus according to claim 5, further comprising a switch configured to output the delayed first synthesized signal to the second synthesizer.

7. The ultrasonic imaging apparatus according to claim 5, wherein the first synthesizer and the second synthesizer generate the first synthesized signal and the second synthesized signal, respectively, by applying a weight value.

8. An Ultrasonic imaging apparatus comprising:
an ultrasonic transducer configured to output a first ultrasonic signal and a second ultrasonic signal; and
a beamforming unit configured to delay and output a first synthesized signal generated using the first ultrasonic signal, and to generate a second synthesized signal by synthesizing the delayed first synthesized signal with the second ultrasonic signal.

9. The ultrasonic imaging apparatus according to claim 8, wherein the first ultrasonic signal is at least one ultrasonic signal output earlier than the second ultrasonic signal.

10. The ultrasonic imaging apparatus according to claim 9, wherein the first synthesized signal is generated by synthesizing a plurality of first ultrasonic signals output earlier than the second ultrasonic signal.

11. The ultrasonic imaging apparatus according to claim 8, wherein the beamforming unit comprises:
a delay unit configured to delay the first ultrasonic signal or the first synthesized signal, and to output the delayed signal; and
a synthesizer configured to synthesize the delayed signal with the second ultrasonic signal to generate the second synthesized signal.

12. The ultrasonic imaging apparatus according to claim 11, wherein the synthesizer synthesizes the delayed signal with the second ultrasonic signal by applying a weight.

13. The ultrasonic imaging apparatus according to claim 11, wherein the delay delays the first ultrasonic signal or the second ultrasonic signal until the second ultrasonic signal is output

14. The ultrasonic imaging apparatus according to claim 11, wherein the beamforming unit further comprises a switch configured to decide a synthesizer to which the delay signal is input.

15. The ultrasonic imaging apparatus according to claim 11, wherein the beamforming unit further comprises an amplifier to amplify the second ultrasonic signal.

16. The ultrasonic imaging apparatus according to claim 11, wherein the beamforming unit further comprises a filter configured to remove noise from the second ultrasonic signal.

17. A method of controlling an ultrasonic imaging apparatus, the method comprising:
delay operation of synthesizing a delayed first ultrasonic signal with a second ultrasonic signal to generate a first synthesized signal, and delaying the first synthesized signal; and
synthesis operation of synthesizing the delayed first synthesized signal with a third ultrasonic signal to generate a second synthesized signal.

18. The method according to claim 17, wherein the delay operation comprises delaying the first synthesized signal until the third ultrasonic signal is output.

19. The method according to claim 17, wherein the synthesis operation comprises applying a weight to the first synthesized signal and the third ultrasonic signal.

20. The method according to claim 19, wherein the synthesis operation further comprises deciding the weight that is to be applied to the first synthesized signal and the third ultrasonic signal.

21. (canceled)
